(19) United States
(12) Patent Application Publication
(10) Pub. No.: US 2021/0222089 A1
(43) Pub. Date: Jul. 22, 2021

(54) COMPOSITION COMPRISING AT LEAST ONE DETERGENT COMPONENT AND AT LEAST ONE ENZYME COMPONENT FOR REMOVING BIOFILMS

(71) Applicants: REALCO, Louvain-la-Neuve (BE); SYMBIOSE BIOMATERIALS, Liège (BE)

(72) Inventors: Gordon Blackman, Lasnes (BE); Sébastien Fastrez, Mons (BE); Camille Gathy, Ceroux-Mousty (BE); Carine Bebrone, Heusy (BE); Hélène Vandegaart, Charneux (BE); Guérin Duysens, Neuville-En-Condroz (BE)

(73) Assignees: REALCO, Louvain-la-Neuve (BE); SYMBIOSE BIOMATERIALS, Liège (BE)

(21) Appl. No.: 16/314,348

(22) PCT Filed: Jun. 29, 2017
(86) PCT No.: PCT/EP2017/066081

§ 371 Date: Dec. 28, 2018

(30) Foreign Application Priority Data
Jun. 29, 2016 (BE) BE2016/5498

(51) Int. Cl. C11D 3/386; C11D 17/04; C11D 3/48; C11D 11/00

(52) U.S. Cl. CPC C11D 3/38636; C11D 17/044; C12N 9/2402; C11D 11/0023; C11D 3/48

(57) ABSTRACT

The invention relates to a composition for removing biofilms, said composition comprising at least one enzyme component and at least one detergent component, the at least one enzyme component containing β-1,6-N-acetylglucosaminidase, and the detergent component containing at least one wetting agent, at least one chelating agent and at least one dispersing agent.

The present invention relates to a composition for removing biofilms, said composition comprising at least one enzyme component and at least one detergent component.

Such a composition is known from document EP2243821 which discloses a composition for removing biofilms present on a substrate, this composition comprising (1) a detergent component simultaneously containing a wetting agent, a sequestering agent and a dispersing agent and (2) comprising an enzyme component simultaneously containing at least one protease, at least one laccase and at least one polysaccharidase. Such a composition is described as being able to be used to remove biofilms in installations, for cleaning-in-place or cleaning-by-soaking floors or surfaces, for cleaning-in-place or cleaning-by-soaking surgical tools and for cleaning in the food industry.

A biofilm is a slime layer which grows on all surfaces, following the adhesion of microorganisms on these surfaces and the secretion therefrom of polymers which coat them and facilitate their adhesion. The biofilms thus form a layer of protection around the microorganisms and represent a recurrent source of contamination of the surrounding environment which poses problems, for example in the food industry and in hospital environments.

More specifically, the accumulation of polymers secreted by the bacteria creates a matrix essentially formed of polysaccharides, DNA, proteins as well as lipids, which protects these microorganisms from external aggressions and is very resistant to conventional cleaning and disinfecting procedures. The microorganisms therefore develop easily within this protective matrix and contaminate the surrounding environment by forming a particularly critical reservoir which is difficult to eliminate.

It is known that the problem of the presence of biofilms is twofold. Firstly, as indicated above, they represent a permanent source of contamination which is very difficult to eliminate by conventional means, even by the most aggressive means. Indeed, common disinfectants are often ineffective as it has been observed that they do not reach the microorganisms which are protected by the biofilm matrix formed of polysaccharides, DNA, proteins and lipids.

Secondly, a biofilm is generally mixed, in that it is initially developed by certain bacterial strains, but it may accommodate others, these strains living and developing in colonies. However, these colonies promote communication between bacteria and, among other things, the exchange and spreading of resistance genes carried by certain bacteria. The biofilms formed by these gene exchanges are thus more difficult to eliminate and increasingly powerful means of disinfection or treatment must be resorted to, which, however, frequently face major problems of resistance and/or tolerance.

The protective matrix of the bacteria forming the biofilms is so resistant that it constitutes a real barrier protecting the bacteria from dehydration phenomena, antibiotic and biocidal action (and more generally, microbicidal molecules), phagocytosis and acids.

In hospital and veterinary environments, but also in agri-food environments, the situation is all the more critical as numerous microorganisms responsible for the formation of biofilms are detected in numerous places. In hospital and veterinary offices, biofilms are detected around individuals (patients and animals) as well as in the surrounding area (operating room, surgical tools, equipment for maintaining said tools, endoscopes, urinary tubes, catheters, medical equipment, dialysis or assisted breathing machines for individuals, etc.) and on surfaces (floors, walls, operating tables, etc.). In the agri-food field, and particularly in factories for manufacturing food products, biofilms can be found around machines, tables, packaging and on operators even if they take all precautions possible in order to avoid contaminating the surfaces and work tools.

From all of this, it appears that biofilms constitute a real problem, particularly in the fields of healthcare (hospitals, dental practices, etc.), veterinary care and agri-food. This problem is all the more critical as biofilms may involve bacteria responsible for infections which could be fatal in individuals, if these bacteria are present in hospitals, veterinary practices or in food products. It is therefore necessary to take all possible precautions in order to avoid the formation and development of biofilms.

These days, in order to fight against biofilms, enzyme formulations comprising a detergent base are being implemented, as proposed in document EP2243821. The majority of the current formulations are based more particularly on a combination of several enzymes, these formulations still typically comprising at least one protease. However, even the formulations (compositions) combining a detergent base and several enzymes have certain limits in terms of removing biofilms from a substrate (from a surface).

Unfortunately, it therefore currently appears that, even if a composition such as that disclosed in document EP2243821 proves to be effective in terms of removing biofilms, the compositions for removing biofilms too often depend on the use of an enzyme cocktail. More specifically, with respect to document EP2243821, the composition for removing biofilms disclosed depends on the use of at least three enzymes (protease and laccase and polysaccaridase) formulated in a detergent base, itself using three different detergent agents (sequestering and wetting and dispersing). It is therefore important, to formulate a composition for removing biofilms according to document EP2243821, to use at least six different components, which offers little economic advantage as handling different components to be formulated together requires time and these six components must be obtained at the same time.

However, it is known that, in a formulation comprising several enzymes including a protease, the latter is inevitably going to gradually deteriorate the other enzymes present over time and forcibly reduce their enzymatic activity over time, this therefore having an impact on the stability and effectiveness of the formulations comprising a protease. Furthermore, it appears that the current compositions have certain limits, particularly with respect to removing “old” biofilms, that is biofilms that have been in place for months or years.

The object of the invention is to overcome the disadvantages of the prior art by obtaining a composition for removing biofilms which is easier to formulate (to use), whose stability and effectiveness are preserved over time and which allows effective action to be taken against all types of biofilm, including “old” biofilms.

To at least partially solve the problems of the prior art, a composition according to the invention is provided for removing biofilms as indicated above, characterised in that said at least one enzyme component comprises β-1,6-N-acetylglucosaminidase and in that said detergent component contains at least one wetting agent, at least one sequestering agent and at least one dispersing agent.

According to the present invention, the enzyme component may comprise β-1,6-N-acetylglucosaminidase as the sole enzyme or the same enzyme combined with others.

It should be noted that, according to EC nomenclature (Enzyme Commission numbers), β-1,6-N-acetylglucosaminidase forms part of the EC 3.2.1-class, in contrast to β-N-acetylglucosaminidase (or hexosaminidase) and β-N-acetylhexosaminidase which form part of the EC 3.2.1.52 class or endo-β-N-acetylglucosaminidase forming part of the EC 3.2.1.96 class.

β-1,6-N-acetylglucosaminidase is active on β-1,6-type bonds while β-N-acetylglucosaminidase (or hexosaminidase) and β-N-acetylhexosaminidase are active on β-1,4-type bonds. In particular, β-1,6-N-acetylglucosaminidase splits the polymer poly-β-1,6-N-acetyl-D-glucosamide (PNAG) which is an exopolysaccharide.

In an entirely surprising and unexpected manner, it has been shown, in the scope of the present invention, that such a composition according to the invention comprising at least β-1.6-N-acetylglucosaminidase in at least one detergent component which comprises at least one wetting agent and at least one sequestering agent and at least one dispersing agent is at least as effective as a composition according to document EP2243821 despite, as indicated above, this composition of the prior art depending on the joint use of three enzymes in the same detergent component. This means that, according to the present invention, an entirely effective composition in terms of removing biofilms from a surface may be prepared by only using β-1,6-N-acetylglucosaminidase in place of an enzyme cocktail depending on the simultaneous presence of three different enzymes. The formulation of a composition according to the invention is consequently easier as it requires the use of fewer different components, which is more economically viable.

In an even more surprising and unexpected manner, it has been determined, within the scope of the present invention, that the three detergent agents of the detergent component have a synergic effect by optimising the effectiveness of β-1,6-N-acetylglucosaminidase in terms of removing the biofilm but also in terms of enzyme activity of the latter over time. On the one hand, it has indeed been determined that this particular combination of three detergent agents (at least one wetting agent+at least one sequestering agent+at least one dispersing agent) ensures significantly higher enzyme activity of β-1,6-N-acetylglucosaminidase over time with respect to the enzyme activities identified for this same enzyme when the detergent component only comprises, for example, two of these three detergent agents. On the other hand, it has been highlighted that this particular combination of three detergent agents increases the effectiveness of the composition according to the invention: optimum removal of the biofilms is observed when β-1,6-N-acetylglucosaminidase is formulated in a detergent component simultaneously comprising at least one wetting agent, at least one sequestering agent and at least one dispersing agent as opposed to in a detergent component only comprising, for example, two of these three detergent agents.

Furthermore, with a composition according to the invention and unlike a composition according to document EP2243821, the presence of a protease is not necessarily required in order to obtain adequate removal of biofilms from the surfaces, this allowing the problem described above to be eliminated, and linked to the presence of this enzyme in a formulation where several enzymes are present.

In addition, unlike the compositions for removing biofilms known in the prior art, it has been demonstrated that a composition according to the invention, comprising at least β-1,6-N-acetylglucosaminidase in at least one detergent component containing at least one wetting agent and at least one sequestering agent and at least one dispersing agent, allows effective action to be taken, even on “old” biofilms which are particularly difficult to remove.

In the scope of the present invention, it has been shown that, following possible detection of the presence of biofilms, for example, using a detection kit as described in document EP2537601, treatment with a composition according to the invention allows β-1,6-N-acetylglucosaminidase to effectively and versatilely deteriorate the organic polymers of different natures forming the matrix of biofilms formed by a multitude of different microorganisms.

Under the action of β-1,6-N-acetylglucosaminidase and together with the action of the detergent component, the biofilm matrix is weakened and inflated, which allows it to be removed from the treated surface. Furthermore, surprisingly, it has also been shown that the composition according to the invention is not specific to a particular microorganism, and thus to a particular type of biofilm, but that it is adapted to numerous bacterial strains.

The detergent action of the composition according to the invention further allows the effectiveness of the composition according to the invention to be ensured. To that end, a detergent base (detergent component) according to the invention is provided which is compatible and which can act in a synergic manner with the enzyme activity of the enzyme component. Furthermore, according to the invention, a detergent base which allows the rapidity and effectiveness of the biofilm removal to be significantly improved is provided. It is for these reasons that the present invention combines a wetting agent and a dispersing agent and a sequestering agent. The joint actions of these three agents of the detergent component of the composition according to the invention allow the surface portion of the biofilm to be removed, to wet and inflate the organic structures of the biofilm in this way, consequently furthering the accessibility of the enzyme component which, in turn, weakens and deteriorates the biofilm matrix.

According to the present invention, the detergent component comprises at least one wetting agent and at least one dispersing agent and at least one sequestering agent. As indicated above, this detergent component acts, first of all, by removing a surface portion of the biofilm, this by wetting and/or inflating the organic structures of the biofilm. In this way, the detergent component furthers the accessibility of the enzyme component by breaking down the biofilm matrix. The enzyme component then acts in synergy with the detergent component and weakens and breaks down the matrix of the biofilm in turn. This action combined with the enzyme component and the detergent component, perfectly compatible with correct action of β-1,6-N-acetylglucosaminidase, furthers the accessibility of the composition to the deeper layers of the biofilms and allows a quick and optimal detachment of any kind of biofilm while preserving the treated substrate (the treated surface). Thus, the detergent component allows the enzymes to act quickly on all biofilm structures.

The dispersing agent of the detergent component allows the separation of the particles of a suspension to be improved in order to prevent agglutination, aggregation and/or decantation. This dispersing agent may be a polymer which is soluble or partially soluble in water such as, for example, polyethylene glycol, cellulose derivatives or a polymer comprising at least one acrylic acid or acrylic ester or polyphosphate unit. Preferentially, the dispersing agent is a polymer comprising at least one acrylic acid or acrylic ester unit of general formula —(CH₂—CH—COOR)—, wherein R represents an alkyl or substituted alkyl, aryl or substituted aryl, or hydrogen group. For example, the dispersing agent is a polymer having an average molecular weight Mw ranging approximately between 500 and 10,000.

More preferentially, the dispersing agent is an acrylic acid polymer. For example, the dispersing agent may be an acrylic acid homopolymer having an average molecular weight ranging approximately between 2,000 and 6,000.

The presence of a dispersing agent in the composition according to the invention thus allows any aggregation of bacterial particles during cleaning of surfaces to be avoided, which ensures an optimal removal of the particles of biofilms detached from a substrate under the action of the enzymes. In fact, rather than joining together, these particles remain separated in a suspension, not re-depositing or re-adhering to the cleaned surface.

The wetting agent of the detergent component is an amphiphilic chemical substance, or a composition comprising said amphiphilic chemical substance, which modifies the surface tension between two surfaces. The wetting agent has the advantage of promoting the spreading of a liquid on a solid but also of increasing the contact between two surfaces. More particularly, the wetting agent has the advantage of furthering contact between the detergent component and a surface and, consequently, between the enzymes and their substrate. For example, on stainless steel surfaces frequently found in the agri-food industries but also in hospital or veterinary environments, the wetting agent allows homogeneous spreading of the composition to be carried out, and thus its perfect distribution on the surfaces to be decontaminated, for example on production tools, workplans, floors, or operating tables and medical tools.

The wetting agent may be anionic, cationic, non-ionic or zwitterionic. Preferentially, the wetting agent may be an anionic or non-ionic wetting agent, that is the hydrophilic part is negatively charged or bears no net charge, or may be a composition comprising an anionic wetting agent. More particularly, the wetting agent may be a sucrose ester or a composition comprising a sodium alkyl sulphate and an alcohol.

The wetting agent may be foaming or non-foaming. Preferably, in the detergent component according to the invention, said wetting agent is not hot-foaming and is preferably chosen from the group of C₆ to C₁₂ sodium alkyl sulphates, C₆ to C₁₀ etheric alcohol sulphates and C₆ to C₁₂ alkyl aryl sulphonates.

The sequestering agent is a chemical substance having the capacity to form complexes with mineral ions which it arranges in a form preventing their precipitation by standard reactions. For example, the sequestering agent may be ethylene-diamine-tetraacetic acid, glucono-delta-lactone, sodium gluconate, potassium gluconate, calcium gluconate, citric acid, phosphoric acid, tartaric acid, sodium acetate, sorbitol, a compound comprising a phosphorous atom. Preferentially, the sequestering agent may be a phosphorous oxide such as phosphonate, phosphinate or phosphate or mixtures thereof, or a salt thereof, an amine or an amine oxide having at least, in its structure, a functional phosphine group, phosphine oxide, phophinite, phosphonite, phosphite, phosphonate, phosphinate or phosphate, alone or in combination, or a salt thereof.

More preferentially, the sequestering agent may be a phosphonate or a salt there of, an amine or an amine oxide comprising at least, in its structure, a functional phosphine group, phosphine oxide, phosphinite, phosphonite, phosphite, phosphonate, phosphinate or phosphate, alone or in combination, or a salt thereof. As a non-limiting example, the phosphonate may be of general formula R¹(R²O)(R³O)P═O, wherein R¹, R² and R³ independently represent a hydrogen, alkyl, substituted alkyl, substituted alkyl-amino or not, substituted amino-alkyl or not, aryl or substituted aryl group. As a non-limiting example, the amine or amine oxide may comprise one, two or three substitute(s) of general formula CR⁴R⁵W, wherein R⁴ and R⁵ represent, independently from one another, a hydrogen, alkyl, substituted alkyl, substituted or non-substituted alkyl-amino, substituted or non-substituted amino-alkyl, aryl or aryl substituted group, and W represents a phosphonate, phosphinate or phosphate group.

The sequestering agent may be in the form of a sodium, calcium, lithium, magnesium or potassium salt; preferentially, the sequestering agent may be in the form of a sodium, calcium or potassium salt.

Preferably, the sequestering agent is an agent which may be used safely in the food industry, in that the sequestering agent is no risk to health, alone or combined with other components.

Advantageously, according to the invention, said at least one enzyme component comprises at least one additional enzyme chosen from the group constituted of α-polysaccharidases (lactase, amylase, α-glucosidase, etc.), β-polysaccharidases (cellulase, hemi-cellulase, β-glucanase, arabanase, pectinase, chitinase, xylanase, dextranase, lysozyme, pullulanase, β-glucisidase, mannanase, etc.), oxidoreductases (laccase, etc.), lyases (lyase pectate, etc.), transferases, proteases and peptidases (metalloprotease, serine-proteases, exopeptidase, endoprotease, cysteine protease, etc.), lipases and esterases (lysophospholipase phospholipase, etc.).

Preferably, the composition for the removal of biofilms according to the invention has a pH ranging between 5 and 11.

In a preferred manner, according to the invention, said at least one detergent component comprises a proportion of sequestering agent between 1 and 10 wt %, a proportion of dispersing agent between 1 and 10 wt % and a proportion of wetting agent between 1 and 30 wt % with respect to the total weight of the detergent component. Preferentially, the proportion of wetting agent between 5 and 20 wt % with respect to the total weight of the detergent component. In a preferred manner, the proportion of wetting agent is 15 wt % with respect to the total weight of the detergent component.

Advantageously, according to the invention, said at least one enzyme component and said at least one detergent component are in solution in a solvent so as to form a vaporisable solution.

Preferentially, according to the invention, said at least one enzyme component and said at least one detergent component are present in solid form, for example, in the form of a lyophilisate, a powder, granules or in any other form soluble in a solvent.

Preferentially, said at least one enzyme component is a solution whose pH may range between approximately 8 and 10.

Preferentially, said at least one enzyme component is an aqueous solution whose pH may range between approximately 5 and 11; more preferentially the pH may be approximately between 7 and 10, and this to preserve the integrity of the enzymes as much as possible.

Alternatively, said at least one enzyme may be in a solid form such as, for example, the form of a lypophilisate, powder, granules or in any other form allowing solubilisation of said component in a solvent, then it will be later dissolved in said solvent. The solvent may be water or an aqueous, acidic, basic, alcoholic, buffer or neutral solution. Said at least one soluble enzyme component could then in this case be later diluted in an aqueous solution optionally containing one or several compounds such as, for example, detergents to form the cleaning solution.

As for said at least one enzyme component, said at least one detergent component may be in a solid form to dissolve in a solvent and/or in an aqueous phase, or in liquid form.

While it is in a solid form, it may either be dissolved directly into the solution formed by the enzyme component optionally already diluted in the aqueous phase, or be dissolved in a solvent, prior to its dilution in the solution formed by the enzyme component and the aqueous phase, or directly in the aqueous phase, before the dilution of the enzyme component.

When said at least one detergent component is in a liquid form, 100% of the detergent components are optionally obtained generally using water and, prior to the application to the biofilm, it will be diluted in an aqueous phase, optionally already containing the enzyme compound.

Other embodiments of a composition according to the invention are indicated in the appended claims.

The object of the invention also includes a method for removing biofilms present on a surface, said method comprising the following steps:
a) providing
   at least one detergent component containing at least one sequestering agent and at least one dispersing agent and at least one wetting agent, and
   at least one enzyme component comprising β-1,6-N-acetylglucosaminidase;
b) dissolving or diluting said at least one detergent component in a solvent;
c) dissolving said at least one enzyme component in the solution formed in step b) to form a composition according to the invention;
or
b′) dissolving or diluting said at least one enzyme component in a solvent,
c′) dissolving said at least one detergent component in the solution formed in step
b′), to form a composition according to the invention,
d) applying said composition formed in step c) or c′) to said surface over a predetermined period of time.

Preferably, according to the method according to the invention, said step of applying said composition is combined with a step of mechanical abrasion of said surface with said composition, for example, by mechanical or manual brushing or by application under medium or high pressure. An additional step of mechanical abrasion allows the solution comprising said composition in aqueous phase to act on the different layers of the biofilms but also to mechanically participate in the breaking down of the polymer matrix and thus to remove the films of the surface of the biofilms so the enzyme and other components of said composition better reach the different layers of the biofilms, which ensures optimal treatment of the surfaces in order to effectively remove the biofilms.

Advantageously, the method according to the invention comprises a last step of applying a biocide to said surface. An additional disinfecting biocide treatment, following the action of the enzyme solution during the step of treating the surface by soaking, allows the destruction of bacteria liberated at the end of the treatment of said surface to be ensured.

Preferably, according to the method according to the invention, said step of applying the solution to said composition is a step taking place over a predetermined period of time ranging between 1 minute and 1 hour, preferably ranging between 3 minutes and 30 minutes, of a solution comprising said composition and a previously formed aqueous dilution phase.

Preferably, the method according to the invention allows a removal of at least 75%, preferably at least 90%, of the microorganisms present on a surface and protected by a biofilm to be ensured.

This microorganism removal rate, of at least 75%, preferably at least 90%, is calculated based on the microorganisms present in a given environment (for example, on the surface of a medical tool or on a floor surface) before and after a step of treating the surface according to the invention.

For example, this microorganism removal rate may be calculated by means of ATP-metric luminometric analysis which allows, by bioluminescence, the number of microorganisms present on a surface to be determined, based on the adenosine triphosphate molecules which were previously removed therefrom, for example, by swabbing. This ATP-metric analysis giving results expressed in relative light units (RLU), the microorganism removal rate is defined according to the following formula: 100−[(RLU value measured after treatment/RLU value measured before treatment)×100].

This microorganism removal rate may also be established by a bacteriological analysis consisting of counting the bacterial colonies on the same surface before and after surface treatment carried out according to the invention. In the case of bacteriological analyses, the microorganism removal rate is calculated according to the following formula: 100−[(CFU value measured after treatment/CFU value measured before treatment)×100].

It is understood that any other technique known to the person skilled in the art and allowing the number of microorganisms before and after treatment of a surface to be determined may also be used in the scope of the present invention.

Other embodiments of the method according to the invention are indicated in the appended claims.

The present invention also includes a kit for removing biofilms present on a surface, said kit comprising:
a sample of at least one detergent component in solution or in solid form containing at least one sequestering agent and at least one dispersing agent and at least one wetting agent; and
a sample of at least one enzyme component in solution or in solid form comprising β-1,6-N-acetylglucosaminidase.

Advantageously, the kit according to the invention further comprises a sample of a biocide in solution or in solid form.

Other embodiments of a kit according to the invention are indicated in the appended claims.

The present invention also relates to a use of a composition according to the invention for removal of biofilms present on a surface by cleaning-in-place, cleaning-by-soaking or by vaporisation of said surface.

The present invention also relates to a use of a kit according to the invention for the removal of biofilms present on a surface by cleaning-in-place, cleaning-by-soaking or by vaporisation of said surface.

Other embodiments of a composition or a kit according to the invention are indicated in the appended claims.

Other features, details and advantages of the invention will become clear from the description given below, in a non-limiting manner and referring to the appended drawings.

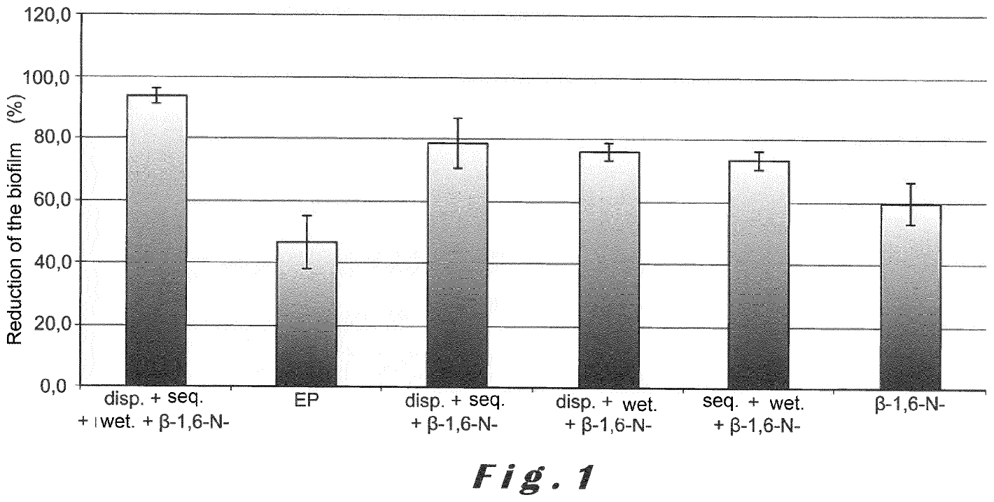

FIG. 1 shows the results obtained in terms of removal of the biofilm (reduction of the biofilm %) formed by Staphylococcus epidermis (EP=composition according to document EP2243821; disp.=dispersing agent; seq.=sequestering agent; wet.=wetting agent; β-1,6-N-=β-1,6-N-acetylglucosaminidase).

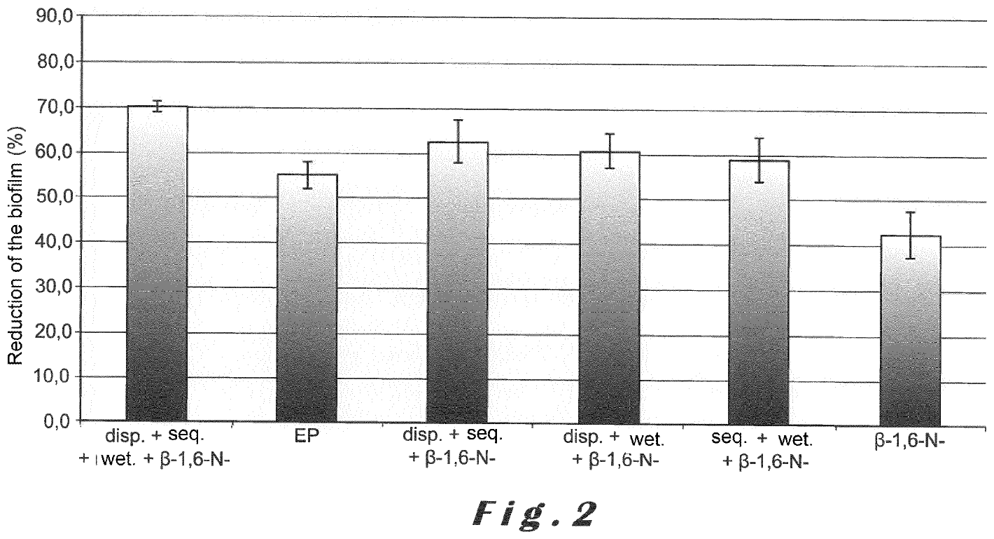

FIG. 2 shows the results obtained in terms of removal of the biofilm (reduction of the biofilm %) formed by Escherichia coli (EP=composition according to document EP2243821; disp.=dispersing agent; seq.=sequestering agent; wet.=wetting agent; β-1,6-N-=β-1,6-N-acetylglucosaminidase).

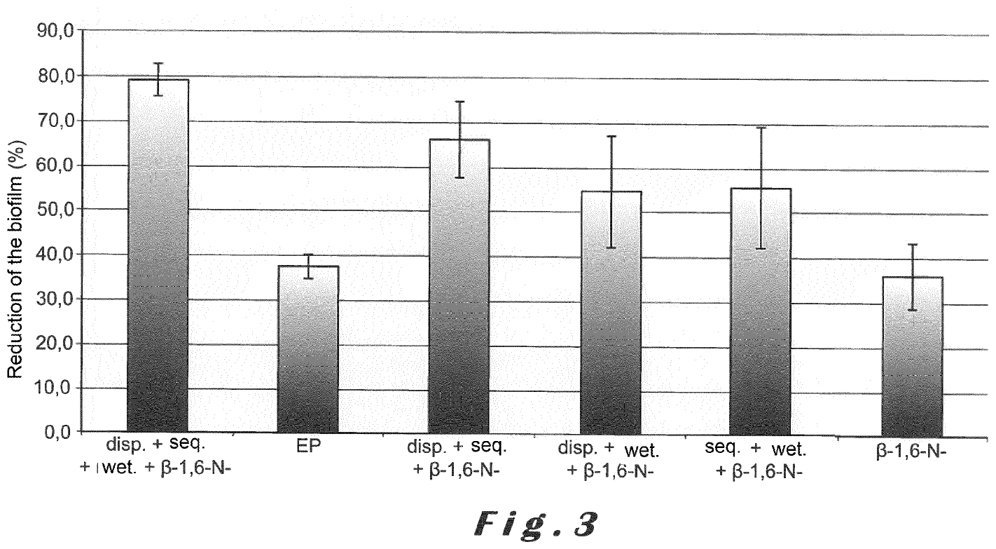

FIG. 3 shows the results obtained in terms of removal of the biofilm (reduction of the biofilm %) formed by Pseudomonas fluorescens (EP=composition according to document EP2243821; disp.=dispersing agent; seq.=sequestering agent; wet.=wetting agent; β-1,6-N-=β-1,6-N-acetylglucosaminidase).

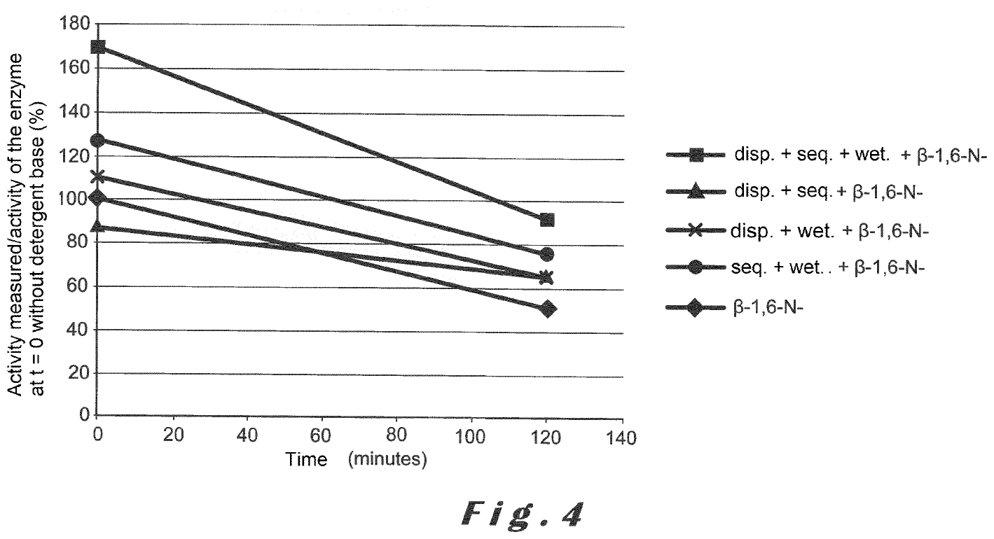

FIG. 4 shows the results of the measurements of the β-1,6-N-acetylglucosaminidase enzyme activities over time based on whether this enzyme is formulated in the presence of three detergent agents (wetting agent+dispersing agent+sequestering agent) or in the presence of two of these three detergent agents or based on whether this enzyme is not formulated in a detergent component.

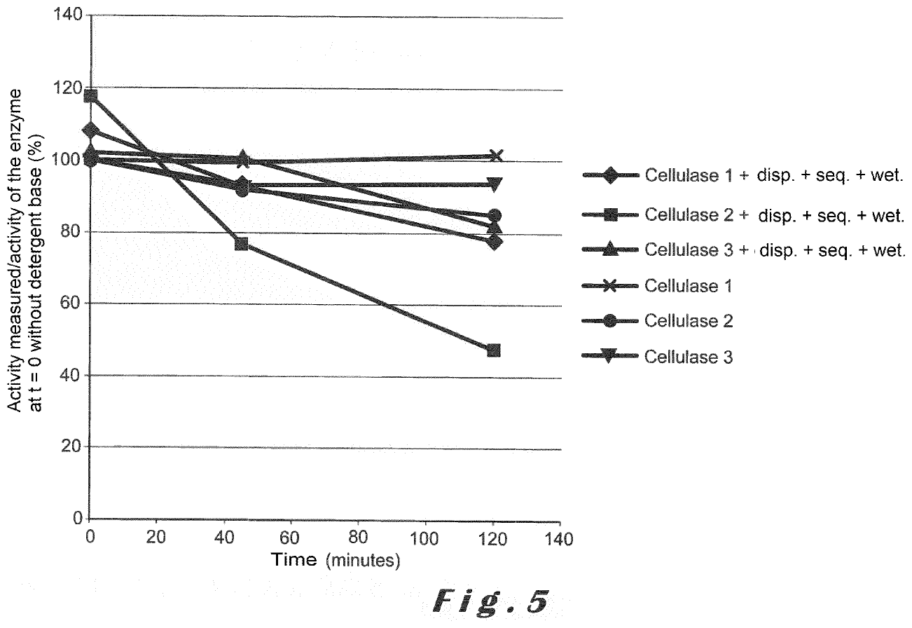

FIG. 5 shows the results of the measurements of the three cellulases over time based on whether or not these enzymes are formulated in the presence of three detergent agents (wetting agent+dispersing agent+sequestering agent) or based on whether this enzyme is not formulated in a detergent component.

### EXAMPLES

### Example 1—Comparative Tests: Effectiveness of a Composition According to the Invention and Effectiveness of a Composition According to Document EP2248321

Comparative tests have been carried out in order to verify that a composition according to the invention is at least as effective in terms of removing a biofilm as a composition such as that disclosed in document EP2243821 (cocktail of 3 enzymes in a detergent component comprising 3 detergent agents).

The composition according to the invention is as follows:
Enzyme component: β-1,6-N-acetylglucosaminidase in an amount of 0.005 v/v % with respect to the total volume of the composition;
Detergent component: Biorem®A1 in an amount of 0.25 v/v % with respect to the total volume of the composition.

The composition according to document EP2243821 is as follows:
Enzyme component: Biorem® 0 in an amount of 0.1 v/v % with respect to the total volume of the composition;
Detergent component: Biorem®A1 in an amount of 0.25 v/v % with respect to the total volume of the composition.

These components are prepared at room temperature by dilution thereof in water before carrying out the comparative tests on a biofilm developed from Pseudomonas fluorescens.

The first steps of the procedure are similar to the steps of the protocol of Annexe F of the ISO 15883 standard. In fact, the protocol of culturing and cleaning the biofilm corresponds to those described in this standard. The number of biofilms present have, in turn, been evaluated using the detection kit according to document EP2243821.

Practically, a biofilm has been developed by Pseudomonas fluorescens in a PTFE (Polytetrafluoroethylene) tube with an internal diameter of 6 mm. The tube was filled with a liquid preculture in the exponential growth phase. The preculture was prepared 24 h before the test: a TSB culture medium was inoculated with the Pseudomonas fluorescens bacterial strain at 30° C. for 12 h. This preculture was then diluted in the TSB culture medium at 10% in order to obtain an optical density ranging between 300 nm and 600 nm. The inoculated culture medium was then circulated in the PTFE tube for 3 days. At the end of this period, the PTFE tube was cut into sections of a size ranging between 5 and 10 cm on which the different compositions were tested. These different compositions were circulated for 15 min at a flow of 100 ml/min at a temperature of 45° C. Finally, the number of biofilms present was evaluated by staining each tube portion with the detection kit disclosed in document EP2537601.

The detection kit is formed of two products, a staining reagent and a cleaning reagent. The blue dye present in the staining reagent specifically adheres to the EPS (extracellular polymeric substances) forming the protective matrix of the biofilm. The surface to be analysed was sprayed with the staining solution. After 5 min of incubation of the surface, the excess solution was absorbed. Then, the surface was rinsed using the cleaning solution of the detection kit which requires an application time of 5 min. The surface was finally rinsed with water and analysed. The presence or absence of biofilm was demonstrated by a blue stain which was darker or lighter according to the number of biofilms existing on the surface. An intense blue stain indicates the significant presence of biofilms.

The results of these tests are presented in Table 1 below.

TABLE 1

| Cleaning | Staining |
|---|---|
| Before cleaning | Intense blue staining |
| Water | Intense blue staining |
| Composition according to document | Absence of blue staining |
| EP2243821 | |
| Composition according to the invention | Absence of blue staining |

As can be seen from these comparative tests, for the tube portions initially strongly contaminated by the biofilm (intense blue staining before cleaning), cleaning with water had no effect on the biofilm and the tube portions initially strongly contaminated by the biofilm consequently retained their intense blue staining.

In contrast, whether in the case of cleaning with the composition according to document EP2243821 or in the case of cleaning with the composition according to the invention, a total removal of the biofilm was ensured (absence of blue staining) after cleaning with these two compositions. This thus indicates that a composition according to the invention allows the biofilms to be removed in a manner at least equivalent to the composition according to document EP2243821 but by only using a single enzyme, i.e. β-1,6-N-acetylglucosaminidase, in place of three enzymes.

### Example 2—Tests of Effectiveness (in Terms of Removal of the Biofilm) of a Composition According to the Invention Depending on Whether the Detergent Component Simultaneously Comprises a Wetting Agent, a Dispersing Agent and a Sequestering Agent or a Combination of Two of these Three Detergent Agents

Effectiveness tests were carried out on the biofilm developed by different bacterial strains, namely Staphylococcus epidermis, Escherichia coli and Pseudomonas fluorescens strains.

The biofilms were developed in laboratories under static conditions in multi-well plates and the effectiveness of β-1,6-N-acetylglucosaminidase (12.5 ppm of the active enzyme in the composition, which corresponds to 0.00125 v/v % with respect to the total volume of the composition) was evaluated on these biofilms following their formulation in the following detergent components:
wetting agent+sequestering detergent+dispersing agent=Biorem®A1 in an amount of 1 v/v % with respect to the total volume of the composition;
wetting agent of Biorem®A1 in an amount of 0.22 v/v % with respect to the total volume of the composition+sequestering agent of Biorem®A1 in an amount of 0.25 v/v % with respect to the total volume of the composition;
wetting agent of Biorem®A1 in an amount of 0.22 v/v % with respect to the total volume of the composition+dispersing agent of Biorem®A1 in an amount of 0.085 v/v % with respect to the total volume of the composition;
sequestering agent of Biorem®A1 in an amount of 0.25 v/v % with respect to the total volume of the composition+dispersing agent of Biorem®A1 in an amount of 0.85 v/v % with respect to the total volume of the composition;

At the same time, β-1,6-N-acetylglucosaminidase (12.5 ppm of the active enzyme in the composition which corresponds to 0.00125 v/v % with respect to the total volume of the composition) was tested alone (not formulated in a detergent component but in aqueous solution) and the effectiveness of a composition according to document EP2243821 (Biorem®A1) at 1 v/v % with respect to the total volume of the composition and Biorem 10® at 0.025 v/v % with respect to the total volume of the composition).

A. Preparation of the biofilms

Bacterial colonies were obtained by separate sowing of each of the strains (Staphylococcus epidermis, Escherichia coli or Pseudomonas fluorescens) in a non-specialised solid medium (PCA) then incubated for 18 h at 37° C. Then, some bacterial colonies (around 20 colonies) were suspended in their respective appropriate culture medium. More particularly, the bacterial colonies were suspended in the following culture media:
Staphylococcus epidermis: suspension of approximately 20 colonies in 2 ml of TGN (30 g/1 of Tryptic Soy Broth+1 g/l glucose+2 gl/NaCl);
Escherichia coli: suspension of approximately 20 colonies in 2 ml of LGB (15 g/l of peptone water+5 g/l yeast extract+10 g/l glucose);
Pseudomonas fluorescens: suspension of approximately 20 colonies in 2 ml of TSB (30 g/1 of Tryptic Soy Broth);

Each of these suspensions were adjusted by addition of culture medium (TGN or LBG or TSB) in order to measure an optical density (O.D.) of 0.05 to 620 nm, then the bacterial suspensions were placed in the wells of multi-well plates in an amount of 100 μl per well for Staphylococcus epidermis, 220 μl per well for Escherichia coli and 220 μl per well for Pseudomonas fluorescens before incubation for 24 h at 37° C. for Staphylococcus epidermis, for 48 h at 30° C. for Escherichia coli and for 48 h at 30° C. for Pseudomonas fluorescens. Naturally, for each of the bacterial strains tested, some wells were only filled with the culture medium in order to form negative controls.

B. Enzyme Treatment of the Biofilms Formed and Quantification of the Removal of the Biofilms

Before treating the biofilms formed with various compositions including a composition according to the invention, the wells of the multi-well plates were emptied of culture media having allowed the establishment and the development of the different biofilms. The treatment of the biofilms itself was carried out by filling the wells with 1 ml of the cleaning solutions (compositions), with softened water being added only to the two positive control wells, the two negative control wells staying empty, in turn. Following these fillings, incubation at 45° C. for 15 min was observed (duration of cleaning to remove the biofilms).

After this incubation period, the wells were emptied of the cleaning solutions and rinsed using a sterile saline solution at 0.9% before drying the plates for 1 h at 60° C. Following this drying, 0.5 ml of crystal violet at 0.1% (powder diluted in demineralised water) was added to each well before incubation for 15 min at room temperature. Following the 15 min of incubation at room temperature, the wells were emptied of the crystal violet solution, rinsed twice with distilled water, then dried with absorbent paper if necessary. 1 ml of acetic acid at 33% was then added to each well before incubation for 1 h at room temperature before undertaking a reading of the plates at a wavelength of 570 nm (measurement of absorbance by spectrophotometry—Thermo Spectronic UV1). The reduction of the biofilm quantity was calculated with respect to an uncleaned control and a control not containing biofilm—the crystal violet (CV) is a cationic dye which will be bound in a non-specific way to the negatively charged constituents of the biofilm and stain them blue. The results of the absorbance measurement of the crystal violet then allow the biofilm still present after cleaning to be quantified.

The results obtained are presented in FIGS. 1, 2 and 3, respectively for the biofilms formed by Staphylococcus epidermis, Escherichia coli or Pseudomonas fluorescens.

As can be seen, whatever the biofilm considered (formed by S. epidermis or by E. coli or by P. fluorescens), a composition according to the invention (β-1,6-N-+disp.+seq.+wet.) allows the biofilms to be removed in a significantly superior way with respect to the other compositions tested. This shows that the three detergent agents have an unexpected synergic effect allowing the action of β-1,6-N-acetylglucosaminidase in terms of removal of the biofilms to be improved. In fact, when β-1,6-N-acetylglucosaminidase is formulated in a detergent component only comprising two of the three detergent agents, it is observed that the removal of the biofilm lessens. The same observation can be made with a composition according to document EP2243821 which does not allow the biofilms to be removed as effectively in comparison with a composition according to the invention.

### Example 3—Enzyme Activity of β-1,6-N-Acetylglucosaminidase Depending on Whether the Detergent Component Simultaneously Comprises a Wetting Agent, a Dispersing Agent and a Sequestering Agent or a Combination of Two of these Three Detergent Agents

The enzyme activity over time of β-1,6-N-acetylglucosaminidase was measured using the 4-nitrophenyl-N-acetyl-b-D-glucosaminide substrate. This substrate releases a yellow compound when it is hydrolysed by β-1,6-N-acetylglucosaminidase. The higher the hydrolysis rate, the higher the concentration of yellow compound and the more active the β-1,6-N-acetylglucosaminidase. The hydrolysis rate is quantified by measuring the absorbance of the solution at 405 nm and allows the enzyme activity of β-1,6-N-acetylglucosaminidase to be judged.

Generally, the enzyme activity over time of β-1,6-N-acetylglucosaminidase was measured according to the following steps:
a) β-1,6-N-acetylglucosaminidase was mixed in the different compositions to be tested (see below) and the thus-obtained solutions were maintained at a temperature of 40° C. for 2 h in order to simulate the cleaning conditions;
b) samples of these solutions were removed at t=0 (t₀) and t=2 h (t_{2 h}) in order to measure the enzyme activity of β-1,6-N-acetylglucosaminidase over time.

More particularly, the following procedure was followed:
a) a buffer solution at 5.9 pH was prepared: 50 mM sodium phosphate+5 mM 4-nitrophenyl-N-acetyl-b-D-glucosaminide+100 mM NaCl;
b) the solution obtained in a) was heated to 37° C. and distributed in an amount of 1 ml in Eppendorfs®;
c) 100 μl of the compositions whose enzyme activity over time must be measured (including a composition according to the invention) was placed in the Eppendorfs® of point b);
d) the concentration of β-1,6-N-acetylglucosaminidase was adjusted by dilution with the phosphate buffer if necessary in order to obtain a concentration of approximately 3.7 μg/ml of β-1,6-N-acetylglucosaminidase;
e) the reaction was stopped by adding 5 μl of 10N NaOH after 5 min;
f) the absorbance was measured at 405 nm (-Thermo Spectronic UV1) after 5 minutes (at t₀+5 min and t_{2 h}+5 min), a negative control (blank) being prepared with water only.

More particularly, the enzyme activity of β-1,6-N-acetylglucosaminidase was measured over time in the following compositions:
wetting agent+sequestering agent+dispersing agent (=Biorem®A1 at 0.25 v/v % with respect to the total volume of the composition)+β-1,6-N-acetylglucosaminidase at 7.4 μg/ml;
wetting agent of Biorem®A1 in an amount of 0.055 v/v % with respect to the total volume of the composition+sequestering agent of Biorem®A1 in an amount of 0.0625 v/v % with respect to the total volume of the composition+β-1,6-N-acetylglucosaminidase at 7.4 μg/ml;
wetting agent of Biorem®A1 in an amount of 0.055 v/v % with respect to the total volume of the composition+dispersing agent of Biorem®A1 in an amount of 0.021 v/v % with respect to the total volume of the composition+β-1,6-N-acetylglucosaminidase at 7.4 μg/ml;
sequestering agent of Biorem®A1 in an amount of 0.0625 v/v % with respect to the total volume of the composition+dispersing agent of Biorem®A1 in an amount of 0.021 v/v % with respect to the total volume of the composition+β-1,6-N-acetylglucosaminidase at 7.4 μg/ml;
β-1,6-N-acetylglucosaminidase at 7.4 μg/ml in aqueous solution.

The results obtained are shown in FIG. 4 and expressed in % of the β-1,6-N-acetylglucosaminidase activity measured for a composition not comprising detergent component (relative enzyme activity). As can be seen, from the start at t₀, that is from the moment when β-1,6-N-acetylglucosaminidase is formulated in a detergent component simultaneously comprising a wetting agent, a sequestering agent and a dispersing agent, its enzyme activity is strongly and significantly potentiated/increased with respect to the enzyme activities measured for β-1,6-N-acetylglucosaminidase when it is formulated in a detergent component only comprising two of the three detergent agents. Subsequently, over time and in particular after 2 h, a significantly superior enzyme activity of β-1,6-N-acetylglucosaminidase is observed for a composition according to the invention with respect to the enzyme activities measured for β-1,6-N-acetylglucosaminidase when it is formulated in a detergent component only containing two of the three detergent agents.

This shows the synergic effect of the three detergent effects which together, in a significant manner, potentiate/increase the enzyme activity of β-1,6-N-acetylglucosaminidase which is, consequently, even more effective over time.

As a comparison with β-1,6-N-acetylglucosaminidase, three cellulases (other enzymes of another family—Celluclast® 1.5l, Carezyme® 4500l, Viscozyme® 120l) were also tested in terms of enzyme activity over time after formulation in Biorem®A1 as detergent component in an amount of 0.25 v/v % with respect to the total volume of the composition.

In particular, the enzyme activity of the three cellulases was measured according to the endo-cellulase procedure (CELLG5® method) of the Megazyme® kit by observing the procedure recommended by the manufacturer. The enzyme activities of the cellulases alone or the cellulases formulated in a detergent component according to the invention were then compared.

The amount of cellulase in the composition was set up in order to be in a concentration range which is in the measurement range of the commercial kit. Preliminary tests were carried out for each of the cellulases tested and an appropriate concentration for measurement was selected for each cellulase.

FIG. 5 shows the results obtained and it can be seen that, in contrast to the results obtained for β-1,6-N-acetylglucosaminidase, the same detergent component (wetting agent+dispersing agent+sequestering agent) absolutely does not have the same effect on the cellulases. In fact, no potentiation/increase of enzyme activity was directly observed following the formulation of each of the cellulases in the detergent component. Furthermore, for these three cellulases, no potentiation/increase of enzyme activity was maintained over time for this detergent component. Quite the contrary, for Cellulase 2, its formulation in a detergent component identical to that of a composition according to the invention significantly decreased the enzyme activity. This further enforced the surprising effect observed with a composition according to the invention where β-1,6-N-acetylglucosaminidase, in contrast to these three cellulases, showed a significantly increased enzyme activity when it was in the presence of three detergent agents (wetting agent+dispersing agent+sequestering agent).

It is understood that the present invention is in no way limited to the embodiments described above and that modifications may be applied without departing from the scope of the appended claims.

1. Composition for the removal of biofilms, said composition comprising at least one enzyme component and at least one detergent component, characterised in that the at least one enzyme component comprises β-1,6-N-acetylglucosaminidase and in that said detergent component contains at least one wetting agent, at least one sequestering agent and at least one dispersing agent.

2. Composition for the removal of biofilms according to claim 1, characterised in that said at least one enzyme component comprises at least one additional enzyme chosen from the group constituted of group constituted of α-polysaccharidases (lactase, amylase, α-glucosidase), β-polysaccharidases (cellulase, hemi-cellulase, β-glucanase, arabanase, pectinase, chitinase, xylanase, dextranase, lysozyme, pullulanase, β-glucisidase, mannanase), oxidoreductases (laccase), lyases (lyase pectate), transferases, proteases and peptidases (metalloprotease, serine-proteases, exopeptidase, endoprotease, cysteine protease), lipases and esterases (lysophospholipase, phospholipase).

3. Composition for the removal of biofilms according to claim 1, characterised in that it has a pH ranging between 5 and 11.

4. Composition for the removal of biofilms according to claim 1, characterised in that said at least one detergent component comprises a proportion of sequestering agent between 1 and 10 wt %, a proportion of dispersing agent between 1 and 10 wt % and a proportion of wetting agent between 1 and 30 wt % with respect to the total weight of the detergent component.

5. Composition for the removal of biofilms according to claim 1, characterised in that said at least one enzyme component and said at least one detergent component are in solution in a solvent, for example, in solution in a solvent in order to form a vaporisable solution.

6. Composition for the removal of biofilms according to claim 1, characterised in that said at least one enzyme component and said at least one detergent component are in a solid form, for example, in the form of a lyophilisate, a powder, granules, or in any other form soluble in a solvent.

7. Method for removing biofilms present on a surface, said method comprising the following steps:
a) providing
at least one detergent component containing at least one sequestering agent and at least one dispersing agent and at least one wetting agent, and
at least one enzyme component comprising β-1,6-N-acetylglucosaminidase;
b) dissolving or diluting said at least one detergent component in a solvent;
c) dissolving said at least one enzyme component in the solution formed in step b) to form a composition according to claim 1;
or
b′) dissolving or diluting said at least one enzyme component in a solvent,
c′) dissolving said at least one detergent component in the solution formed in step b′), to form a composition according to claim 1,
d) applying said composition formed in step c) or c′) to said surface over a predetermined period of time.

8. Method according to claim 7, characterised in that said step of applying said composition is combined with a step of mechanical abrasion of said surface with said composition by mechanical or manual brushing or by application under medium or high pressure.

9. Method according to claim 7, characterised in that it comprises a last step of applying a biocide to said surface.

10. Kit for the removal of biofilms present on a surface, said kit comprising:
a sample of at least one detergent component in solution or in a solid form containing at least one sequestering agent and at least one dispersing agent and at least one wetting agent; and
a sample of at least one enzyme component in solution or in a solid form comprising β-1,6-N-acetylglucosaminidase.

11. Kit according to claim 10, characterised in that it further comprises a sample in solution or in a solid form of a biocide.

12. Use of a composition according to claim 1 for removal of biofilms present on a surface by cleaning-in-place, cleaning-by-soaking or cleaning-by-vaporisation of said surface.

13. Use of a kit according to claim 10 for removal of biofilms present on a surface by cleaning-in-place, cleaning-by-soaking or cleaning-by-vaporisation of said surface.
